(19) United States
(12) Patent Application Publication
(10) Pub. No.: US 2012/0141446 A1
(43) Pub. Date: Jun. 7, 2012

(54) Oral Nutritional Supplement Delivery System

(76) Inventor: Bhiku Patel, Chandler, AZ (US)

(21) Appl. No.: 12/958,439

(22) Filed: Dec. 2, 2010

(51) Int. Cl. A61K 31/122; A23L 1/303; A61K 31/59; A61K 31/202; A61K 36/82; A61K 31/045; A61P 3/10; A61P 7/00; A61P 19/08; A61P 19/02; A61P 25/00; A61P 37/02; A61P 35/00; A61P 9/00; A23L 1/30

(52) U.S. Cl. 424/94.1; 426/601; 426/73; 426/2; 514/167; 514/560; 424/729; 514/724

(57) ABSTRACT

A composition for increasing the bioavailability of nutrients in humans and animals comprising an oil base emulsion into which nutrients selected from the group comprising omega fatty acids, vitamin D, Co Q10, green tea extract, and policosanol are added.

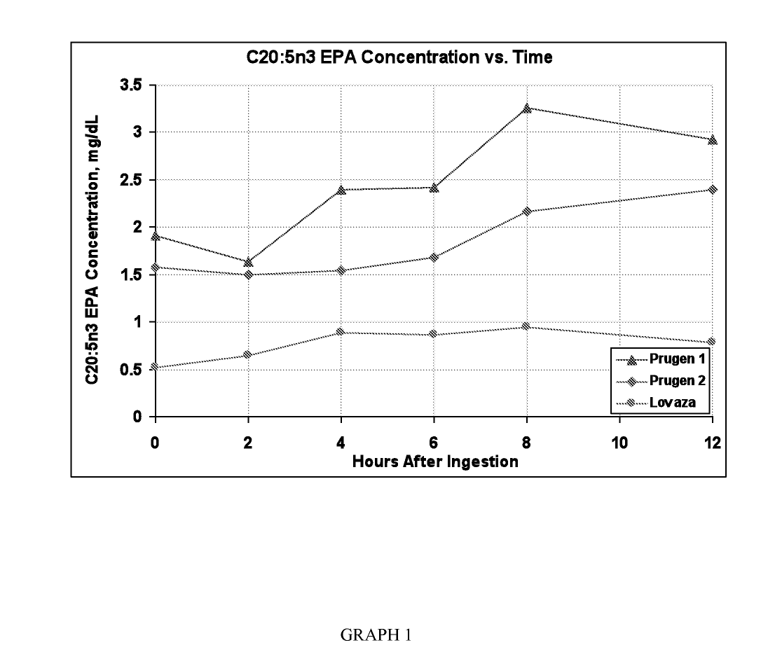

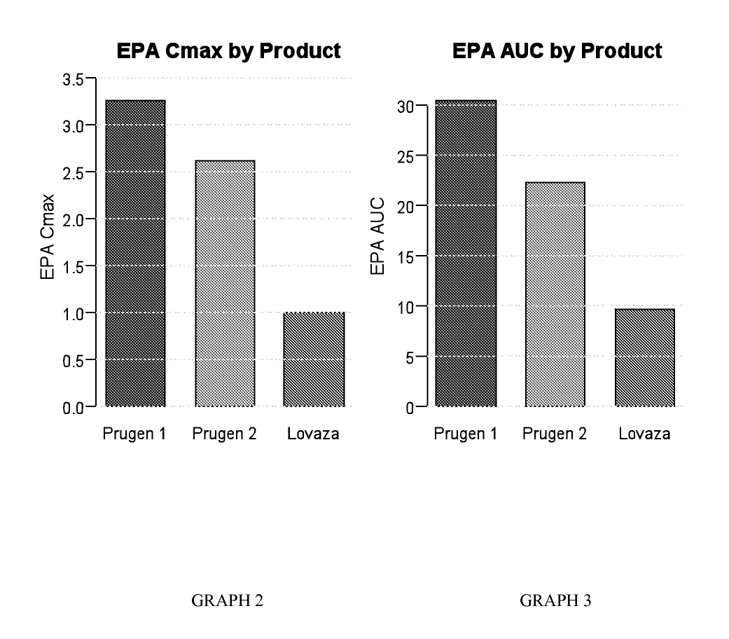

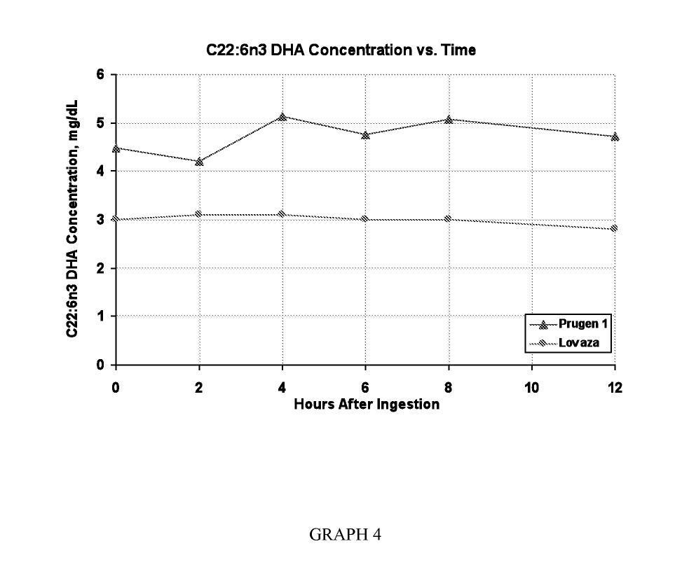

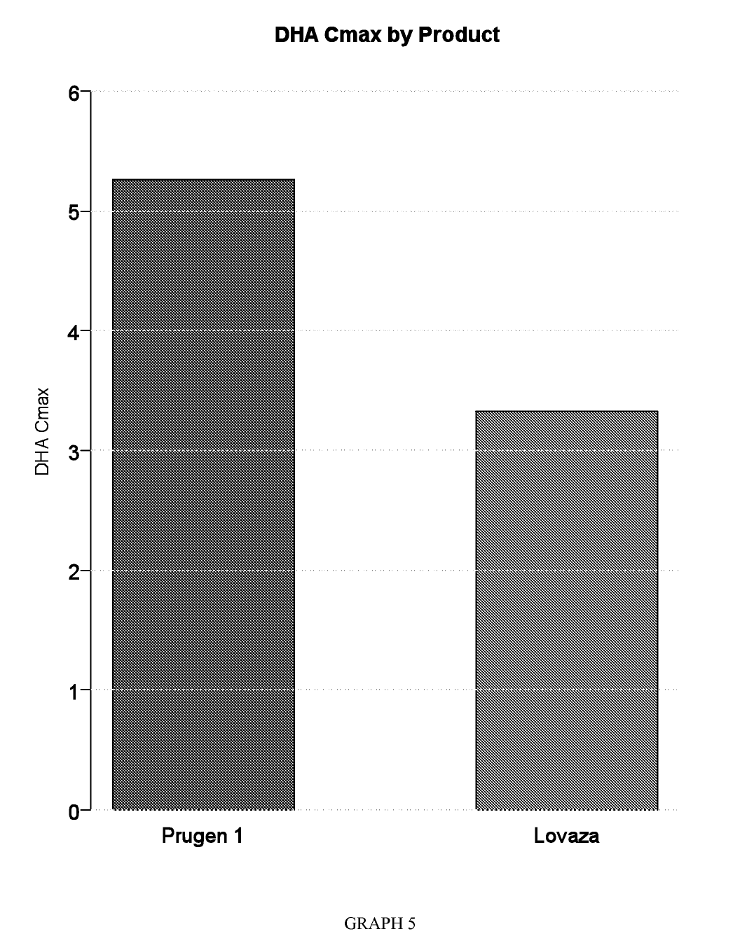

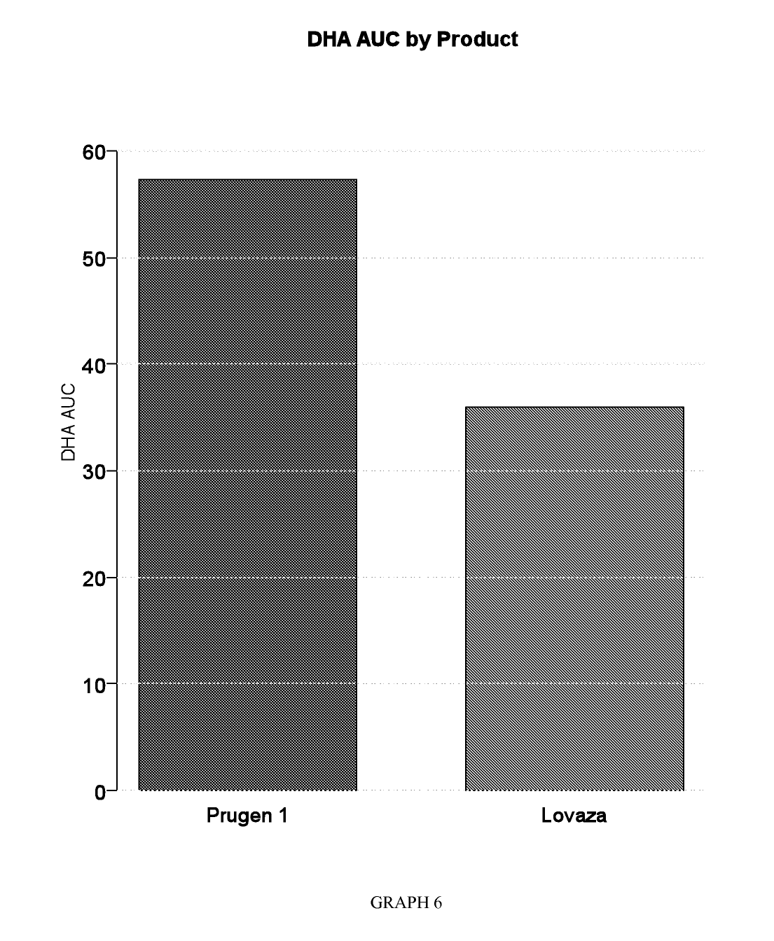

### I. INCORPORATION BY REFERENCE

This application incorporates by reference the full content of application Ser. No. 12/906,419 filed Oct. 18, 2010.

### II. TECHNICAL FIELD

The present invention relates to oral nutritional supplements and, more particularly, to a composition that provides for a combination of nutritional supplement ingredients to be delivered through an enhanced bioavailability mechanism.

### III. BACKGROUND OF THE INVENTION AND PRIOR ART

Nutritional products are well known and generally accepted. They run the gambit from vitamins and minerals to more exotic substances. Nutritional products are currently sold for general health support and cannot be marketed for the diagnosis or treatment of any disease. It is generally accepted, however, that certain nutritional products do play a role in supporting certain bodily functions or as medical treatment adjunct therapies, notably cardiovascular health. Among these nutrients are omega fatty acids, vitamin D, Co Q10, green tea extract, and policosanol. Recent studies have also shown promise in the use of these nutrients variously for controlling blood pressure, aiding in the treatment of diabetes mellitus, and assisting in bone structure maintenance. There is further evidence mounting for the use of them in other areas of health, including, for example, brain function, arthritis, immune system maintenance, and cancer prevention.

Omega fatty acids, especially docosahexaenoic acid (DHA) and eicosapentaenoic acid (EPA), are generally accepted as promoting good heart health. Many healthcare practitioners recommend that their patients take an omega fatty acid supplement. These fatty acids are what are known as essential fatty acids (“EFA”). Omega fatty acids are not produced by the body; they must be obtained from other sources. These essential fatty acids are found in many sources, including fish and crustacean marine sources, plants, algae, and animals. There is no set dosage for omega fatty acids.

Depending on source of the omega fatty acids (purity) and patient health needs, dosing is recommended at between 1000 mg and 6000 mg. The American Heart Association currently recommends that heart patients take 1000 mg per day.

Recent research has also demonstrated that vitamin D plays a more significant role in overall health than previously recognized. In addition to the well-documented need for vitamin D in bone health, several studies have confirmed the importance of vitamin D in, for example, controlling blood pressure, supporting cognitive function, boosting immunity, regulating insulin secretion, maintaining calcium balance for neurological function, and providing protection against certain cancers.

There are two forms of vitamin D used by the body, ergocalciferol (vitamin D2) and cholecalciferol (vitamin D3), with vitamin D3 being thought to be the more important. Historically, the recommended daily dosage of vitamin D was 400 IU. More recent research has shown, however, that levels of at least 2000 IUs are necessary to raise the blood level of vitamin D with some researchers recommended as much as 4000 IUs daily in order to gain maximum benefit.

Co Q10 is another example of a nutrient that may play a more significant role in general health than previously thought. Co Q10, or more properly Coenzyme Q10 or ubiquinone, is a naturally ocurring component of the electron transport chain; it participates in aerobic cellular respiration. Co Q10 is an oil-soluble substance that is present in most eukaryotic cells, primarily in the mitochondria. Necessary for energy production, and therefore overall health, Co Q10 is critical, however its production is inhibited by some beta blockers, blood pressure-lowering medication, and statins, a class of cholesterol-lowering drugs. Western diet, principally in food preparation, is thought to deplete or inhibit Co Q10. Current thought is to use a Co Q10 supplement as adjunct therapy for drugs known to inhibit it productions, especially the statins. There is no set standard for Co Q10 dosing but general thought is 30 mg to 200 mg daily, depending on overall health and medication intake, is appropriate.

Another nutritional supplement that has gained acceptance is green tea extract. Green tea extracts are herbal derivatives containing antioxidant ingredients, mainly catechins Anti-oxidants scavenge for free radicals thereby reducing inflammation and providing some protection against cellular division breakdown. Studies have demonstrated that green tea extracts exhibit stronger antioxidant protection for human body than vitamin C and vitamin E. In fact, green tea extract is 20 times more antioxidant-active than Vitamin C.

Green tea extracts' anti-inflammatory characteristics may make it useful in treating chronic inflammatory states, improving cardiovascular health, reducing blood pressure. Its anti-oxidative properties, in addition to the foregoing, shows promise in providing protection against certain cancers and is also thought to help slow the aging process. Green tea extracts are also touted as assisting in weight-loss. Though there is set dosing for green tea extract, a daily dose of 300 to 400 mg green tea extracts is recommended by many experts.

Policosanols are another nutritional supplement that have been of increased interest of late. Policosanols are aliphatic alcohols found in sugar cane, yams, beeswax and certain other insect secretions. Studies, principally out of Cuba, have shown policosanol to lower LDLs (bad cholesterol) and raise HDLs (good cholesterol). Other studies, however, have shown policosanol to have no such effects. Despite this contradictory information, consumers have exhibited an interest in supplementing their diets with policosanol.

A drawback of these nutritional products is that it currently requires an individual to ingest numerous separate tablets, capsules, or gelcaps to obtain proper levels of the nutrients. From a treatment standpoint, having to take multiple supplements (perhaps in addition to multiple medications) makes compliance an issue. Further, from a pure scientific standpoint there is interest in making as much of the nutrients bioavailable as possible so as to get the maximum effect in a short amount of time.

Attempts to address these issues have, until the present invention, have, with the exception of Co Q10, focused on increasing the amount of a nutrient, not, necessarily, on making a more palatable and easy to use regimen. And while vitamin D is found in multivitamins, there has been no attempts to combine it with the other described nutrients in order to provide a single highly bioavailable supplement that is designed to support the known and suggested properties of these particular nutrients.

With regard to Co Q10, attempts have been made to boost its bioavailability after oral administration. One attempted strategy is to reduce the particle size to as low as the micro- and nano-scale. However, this approach has not yet proved to be very successful.

Another approach is to put Co Q10 into an emulsion to facilitate absorption from the gastrointestinal tract and to improve bioavailability. This attempt included the use of soybean oil emulsions of soybean oil. Using this system, Ozawa et al. (Y. Ozawa, Y. Mizushima, I. Koyama, M. Akimoto, Y. Yamagata, H. Hayashi and H. Murayama, Drug Res., 36-1: 689-690 (1986)) performed a pharmacokinetic study on beagle dogs in which the emulsion of CoQ10 in soybean oil was investigated; about two times higher plasma CoQ10 level than that of the control tablet preparation was determined during administration of a lipid microsphere. However, Kommuru et al. (T. R. Kommuru, M. Ashraf, M. A. Khan and I. K. Reddy, Chemical & Pharmaceutical Bulletin, 47: 1024-1028 (1999), in a similar experiment, observed almost no increase in Co Q10 with oil-based soft-gel capsules in a later study on dogs.

Various approaches have also been developed to increase Co Q10 water solubility. Many of them have produced significantly better results over oil-based soft-gel capsules. Examples of such approaches are use of the aqueous dispersion of solid CoQ10 with tyloxapol polymer, formulations based on various solubilising agents, i.e., hydrogenated lecithin, and complexation with cyclodextrins.

Another concern with many nutrients, not just those discussed above, is that most of the nutrient ingested is not absorbed but, instead, excreted. This has raised concern by healthcare professionals and the public that taking nutrient supplements may be either a waste of time and money or ineffective.

Accordingly, there is need for a composition that increases the bioavailability of certain nutrients in an oil based environment while at the same time shortening the time for absorption, and potentially reducing the amount of nutrient necessary to be consumed in order to obtain a desired result. Such a composition would provide improve therapy support, increased levels of selected nutrients, and ease of use for a patient. Such a composition is provided for in the present invention.

### IV. OBJECTS AND ADVANTAGES OF THE PRESENT INVENTION

It is an object of the present invention to provide a composition that enhances the bioavailability of a consumed nutrient in an oil based solution.

It is further an object of the present invention to provide a composition that shortens the time of absorption of such a composition.

It is further an object of the present invention to provide a finely dispersed oil based solution containing nutrient companents.

It is further an object of the present invention to enable the reduction in the amount of nutrient to be consumed.

The advantages offered by the present invention include but are not limited to maximizing the amount of bioavailable consumed nutrients.

### V. SUMMARY OF THE INVENTION

The present invention comprises a composition containing various combinations of nutrients selected from a group comprising omega fatty acids, vitamin D, Co Q10, green tea extract, and policosanol in an oil based solution for humans and animals that enables increased bioavailability of the nutrients. The oil base comprises a first emulsifier and a second emulsifier in a ratio ranging from about 1:1 to about 4:1, preferably, the first emulsifier and the second emulsifier are mixed in a ratio of about 2:1. Preferably, the first emulsifier is polyoxyethylene sorbitan monooleate and the second emulsifier is tocopheryl polyethylene glycol succinate. Selected nutrients are then added to the oil base with the final mixture of the ingredients being in a ratio ranging from about 99:1 to about 9:1. The base oil can be made from any consumable oil such as marine, plant, or animal oils.

The nutrients can be added to the oil base in any combination, depending on the requirements of the user. For instance, each of omega fatty acids, vitamin D, Co Q10, green tea extract, and policosanol can be added to the oil base or, alternatively, only omega fatty acid, vitamin D, and Co Q10 could be added. The combination is not critical. It is only important that nutrients selected are placed in the oil base in therapeutically effective amounts as required by the user.

There has been outlined, rather broadly, the more important features of the invention in order that the detailed description thereof that follows may be better understood, and in order that the present contribution to the art may be better appreciated. There are, of course, additional features of the invention that will be described hereinafter and that will form the subject matter of the invention.

### VI. BRIEF DESCRIPTION OF THE DRAWINGS

Graph Number 1 depicts EPA blood level Concentration vs Time (Hrs) for the inventive composition compared with purified fish oil alone and with a prescription fish oil based medication.

Graph Number 2 depicts the maximum EPA blood level concentration for the inventive composition compared with purified fish oil alone and with a prescription fish oil based medication.

Graph Number 3 depicts the maximum EPA AUC for the inventive composition compared with purified fish oil alone and with a prescription fish oil based medication

Graph Number 4 depicts DHA blood level Concentration vs Time (hrs) for the inventive composition compared with prescription fish oil based medication.

Graph Number 5 depicts the maximum DHA blood level concentration for the inventive composition compared with prescription fish oil based medication.

Graph Number 6 depicts the maximum DHA AUC for the inventive composition compared with a prescription fish oil based medication.

### VII. DETAILED DESCRIPTION OF THE PREFERRED EMBODIMENT

Before explaining the preferred embodiment of the present invention in detail, it is to be understood that the present invention is not limited in its application to the details of formulations and arrangements of the components set forth in the following description. The present invention is capable of other embodiments and of being practiced and carried out in various ways. Also, it is to be understood that the phraseology and terminology employed herein are for the purpose of description and should not be regarded as limiting. It is also to be understood that where ranges are provided for the various agents and drug examples, they are approximate ranges and are not to be limiting except where noted otherwise.

The present invention addresses the problem of providing therapeutic amounts, in various combinations, of omega fatty acids, vitamin D, Co Q10, green tea extract, and policosanol in a single, easy to use form. To overcome limitations in absorption and bioavailability of these nutrients, the Inventor has discovered that dispersing them in a finely dispersed micro-emulsion in turn creates an increase in surface area per volume, thereby enabling greater gastrointestinal uptake of the nutrients. The Inventor has found that through the use of emulsifiers that do not create a permanent binding to the nutrients, their bioavailability is increased.

To create a micro-emulsion adequate to provide the necessary results, the Inventor discovered that two emulsifiers are required. A first emulsifier should be a nonionic surfactant and emulsifier. There is a wide range of such emulsifiers from which to choose. Key to the selection is that the first emulsifier be suitable for human or animal ingestion (throughout, the terms consumption and ingestion are used interchangeable and mean to take orally.) The Inventor has discovered that the preferred first emulsifier is polyoxyethylene sorbitan monooleate.

A second emulsifier is also required. The Inventor has discovered that employing esterfied Vitamin E works well. While esterfied Vitamin E in its various forms may be employed in the invention, the Inventor has discovered that the preferred form is tocopheryl polyethylene glycol succinate.

Both of the preferred emulsifiers are well known in the industry. Polyoxyethylene sorbitan monooleate has been used for many years in the food and pharmaceutical industries. It is most commonly sold under the trade name polysorbate 80 and is widely available. It is approved by the U.S. Food and Drug Administration as an inactive ingredient and is well tolerated in oral compositions. Tocopheryl polyethylene glycol succinate is sold by several companies but was first developed by the Eastman Company and sold under the trademark ‘Vitamin E TPGS NF.’ It was developed as a water soluble emulsifier to aid in the absorption of lipid-based drugs, such as cyclosporin. Since its invention, it has been used in many products.

However, until the present invention the combination use of polyoxyethylene sorbitan monooleate and tocopheryl polyethylene glycol succinate to enhance the bioavailability of certain nutrients has not been taught.

Individually, each of these emulsifiers will cause at least some dispersion but it is the heretofore unknown mixture of the two emulsifiers and oil that creates the micro-emulsion necessary to increase the bioavailability of the oil. Using the Inventive Composition, a fine micro-emulsion is created that enables almost complete dispersion in water, a result unseen with either of the two emulsion separately or known in the prior art. The result is gained by the emulsion properties of the tocopheryl polyethylene glycol succinate to create a fine dispersion and the polyoxyethylene sorbitan monooleate to reduce further the interfacial tension that then enables an oil-in-water micro-emulsion of a level not heretofore seen in the art.

When considering the selection of emulsifiers to use in combination to create a suitable micro-emulsion, the Inventor discovered that surface activity of the emulsifiers on the oil when in combination with the nutrients was an important element. Increasing the surface area of the oil enabled increased bioavailability but only to a point. Once surface area exceeds a certain value, no additional benefit is gained in absorption and, therefore, bioavailability. In fact, bioavailability can be decreased.

To determine the suitable range of surface area activity, the Inventor employed hydrophilic lipophilic balance (HLB) values. HLB is a widely accepted method for providing a measure of the surface activity of organic molecules. HLB values for emulsifiers range from about 2 to about 40. The Inventor discovered that emulsifiers in the range of about 10 to about 30 are suitable for use with the invention, with a range of about 12 to about 16 providing the best results.

Polyoxyethylene sorbitan monooleate has an HLB value of about 15 and tocopheryl polyethylene glycol succinate has an HLB value about 13. Thus, the average HLB value for the combination of the two emulsifiers is about 14 when they are found in a 1:1 polyethylene glycol succinate: tocopheryl polyethylene glycol succinate ratio and about 14.6 when they are in a 4:1 polyethylene glycol succinate: tocopheryl polyethylene glycol succinate ratio. Accordingly, the preferred HLB range is from about 14 to about 14.6.

To demonstrate the effectiveness of the combination of these two emulsifiers in an oil, the Inventor developed an experiment to illustrate the increased bioavailability of omega fatty acids, using EPA and DHA as markers. In the experiment, subjects were cleared of blood stream detectable levels of EPA and DHA. The Human subjects were then randomly given either the Inventive composition, a purified fish oil, or a fish oil-based prescription medication under physician supervision. Blood level readings for both EPA and DHA were then taken at 2, 4, 6, 8, 10, and 12 hours.

Turning to Graph Number 1, it can be seen that EPA levels for the inventive composition were significantly above those for both the purified fish oil and the prescription medication at every reading. The following table 1 illustrates the rate of blood level concentration (mg/dl/hr) for each of the three test materials:

TABLE 1

| Inventive Composition | Purified Fish oil | Prescription Medication |
|---|---|---|
| 0.253 | 0.10 | 0.06 |

Turning to Graph Number 2, it can be seen that the maximum blood concentration (C.) for the Inventive Composition EPA is also greater than that of the purified fish oil and the prescription medication. The following table 2 shows the Cₘₐₓ (mg/dl) for each of the three test materials:

TABLE 2

| Inventive Composition | Purified Fish oil | Prescription Medication |
|---|---|---|
| 3.27 | 2.62 | 1.00 |

Graph Number 3 illustrates the Area Under Curve (AUC) for the three test materials and, again demonstrates the superiority of the Inventive Composition. Table 3 shows the average AUC₀₋₁₂ (mg/d1/hr) for the three test materials:

TABLE 3

| Inventive Composition | Purified Fish oil | Prescription Medication |
|---|---|---|
| 30.40 | 22.30 | 9.70 |

DHA readings were then taken to further demonstrate the Inventive Composition effectiveness. Turning to Graph 4, it can be seen that DHA levels for the inventive composition were significantly above those of the prescription medication at every reading.

Turning to Graph Number 5, it can be seen that the maximum blood concentration (C.) for the Inventive Composition DHA is also greater than that of the prescription medication. The following table 4 shows the Cₘₐₓ (mg/dl) for the two test materials:

TABLE 4

| Inventive Composition | Prescription Medication |
|---|---|
| 5.26 | 3.34 |

Graph Number 6 illustrates the Area Under Curve (AUC) for the two test materials and, again demonstrates the superiority of the Inventive Composition. The AUC₀₋₁₂ for the Inventive Composition is 57.3 mg/dl/hr. and for the prescription medication is 33.0 mg/dl/hr.

In mixing of the two emulsifiers with oil to create a more bioavailable oil, the ratios of the two emulsifiers and oil in creating an end product with maximum effectiveness at a minimum use of emulsifiers have been considered. The Inventor has discovered that an emulsifier ratio in the range of polyoxyethylene sorbitan monooleate:tocopheryl polyethylene glycol succinate of about 1:1 to about 4:1 is useful. The preferred ratio is 2:1.

Once the oil base is created, nutrients can be dispersed in solution. Omega fatty acids, vitamin D, Co Q10, green tea extract, and policosanol can be added to the oil base in any combination. When dispersed into the oil base they temporarily bind to the oil and are absorbed in the gastrointestinal tract at an increased level as compared to when ingested in other forms.

In mixing the components together, the emulsifier combination, which can be done as a separate step and added to the oil or individually added to the oil, the Inventor has discovered that the final mixture of oil:emulsifiers should be about 99:1 to about 9:1. Levels above 99:1 do not allow adequate dispersion to take place and level below 9:1 cause gelling of the oil-emulsifier combination. The preferred ratio is about 12.333:1

The nutrients can be added to the oil before it is mixed with the emulsifiers or they can be added after the oil is emulsified. The Inventor has found, however, that a better dispersion is achieved when the nutrients, with the exception of the omega fatty acids, are added after the oil emulsification step. While omega fatty acids can also be added after the emulsification step, often these acids are found in the oil selected for the base. Therefore, they are added to the combination simultaneously with the oil.

The nutrients should be added to the oil base in therapeutically effective amounts. For example, omega fatty acids could be added in an amount of 600 mg to 6000 mg, vitamin D up to an amount of 4000 IU, Co Q10 up to 300 mg, and policosanol and green tea extract as needed.

Once mixed and dispersed, the combination can be placed in a liquid or gelcap form. While either form increases the bioavailability of each of the nutrients, the use of a gelcap form is best for the well-known reason that the gelcap will dissolve further into gastrointestinal tract, thereby enabling an even greater amount of nutrient absorption. Enteric coated gelcaps provide even more time until the cap dissolves than a plain gelcap.

It is to be understood, however, that even though numerous characteristics and advantages of the preferred and alternative embodiments have been set forth in the foregoing description, together with details of the structure and function of the embodiments, the disclosure is illustrative only, and changes may be made in detail within the principles of the invention to the full extent indicated by the broad general meaning of the terms in which the appended claims are expressed.

I claim:

1. A composition for increasing the bioavailability of nutrients in humans and animals comprising a combination of therapeutically effect amount of nutrients dispersed into an oil base emulsification having a first emulsifier and a second emulsifier in a ratio ranging from about 1:1 to about 4:1 with a consumable oil.

2. The composition of claim 1 wherein the first emulsifier and the second emulsifier are mixed in a ratio of about 2:1.

3. The composition of claim 1 wherein the nutrients are selected, either singly or in combination, from the group comprising omega fatty acids, vitamin D, Co Q10, green tea extract, and policosanol.

4. The composition of claim 1 wherein the first emulsifier is polyoxyethylene sorbitan monooleate.

5. The composition of claim 1 wherein the second emulsifier is tocopheryl polyethylene glycol succinate.

6. The composition of claim 1 wherein the consumable oil is selected, either singly or in combination, from the group comprising animal oils, vegetable oils, marine-based oils, and algae oils.

7. The composition of claim 1 wherein the first emulsifier and the second emulsifer have individual HLB values in the range of about 10 to about 30.

8. The composition of claim 7 wherein the first emulsifier and the second emulsifer have individual HLB values in the range of about 12 to about 16.

9. The first emulsifier and the second emulsifier of claim 7 wherein the first emulsifier and second emulsifier, when in combination, have an HLB value in the range of about 14 to about 14.6.

10. A composition for increasing the bioavailability of nutrients in humans and animals comprising mixing polyoxyethylene sorbitan monooleate and a second emulsifier in a ratio ranging from about 1:1 to about 4:1 with a consumable oil.

11. The composition of claim 10 wherein the polyoxyethylene sorbitan monooleate and the second emulsifier are mixed in a ratio of about 2:1.

12. The composition of claim 10 wherein the consumable oil is admixed with a mixture of the first emulsifier and the second emulsifier in a ratio ranging from about 99:1 to about 9:1.

13. The composition of claim 12 wherein the ratio is about 12.333:1.

14. The composition of claim 13 wherein the second emulsifier is tocopheryl polyethylene glycol succinate.

15. The composition of claim 13 wherein the consumable oil is selected, either singly or in combination from the group comprising animal oils, vegetable oils, marine-based oils, and algae oils.

16. The composition of claim 10 wherein the nutrients are selected, either singly or in combination, from the group comprising omega fatty acids, vitamin D, Co Q10, green tea extract, and policosanol.

17. The composition of claim 10 wherein the first emulsifier and the second emulsifer have individual HLB values in the range of about 10 to about 30.

18. The composition of claim 10 wherein the first emulsifier and the second emulsifer have individual HLB values in the range of about 12 to about 16.

19. The first emulsifier and the second emulsifier of claim 10 wherein the first emulsifier and second emulsifier, when in combination, have an HLB value in the range of about 14 to about 14.6.

20. A composition for increasing the bioavailability of nutrients in humans and animals comprising mixing a first emulsifier and tocopheryl polyethylene glycol succinate in a ratio ranging from about 1:1 to about 4:1 with a consumable oil.

21. The composition of claim 20 wherein the first emulsifier and the tocopheryl polyethylene glycol succinate are mixed in a ratio of about 2:1.

22. The composition of claim 20 wherein the consumable oil is admixed with a mixture of the first emulsifier and the second emulsifier in a ratio ranging from about 99:1 to about 9:1.

23. The composition of claim 22 wherein the ratio is about 12.333:1.

24. The composition of claim 20 wherein the first emulsifier is polyoxyethylene sorbitan monooleate.

25. The composition of claim 20 wherein the consumable oil is selected, either singly or in combination from the group comprising animal oils, vegetable oils, marine-based oils, and algae oils.

26. The composition of claim 20 wherein the nutrients are selected, either singly or in combination, from the group comprising omega fatty acids, vitamin D, Co Q10, green tea extract, and policosanol.

27. The composition of claim 20 wherein the first emulsifier and the second emulsifer have individual HLB values in the range of about 10 to about 30.

28. The composition of claim 20 wherein the first emulsifier and the second emulsifer have individual HLB values in the range of about 12 to about 16.

29. The first emulsifier and the second emulsifier of claim 20 wherein the first emulsifier and second emulsifier, when in combination, have an HLB value in the range of about 14 to about 14.6.

30. A method for increasing the bioavailability of nutrients comprising the steps of:
mixing a first emulsifier and a second emulsifier in a ratio ranging from about 1:1 to about 4:1 with a consumable oil an oil base;
adding at least one nutrient to the oil base; and,
consuming the first emulsifier, second emulsifier, and oil combination.

31. The method of claim 30 including the step of selecting the first emulsifier as polyoxyethylene sorbitan monooleate.

32. The method of claim 30 including the step of selecting the second emulsifier as tocopheryl polyethylene glycol succinate.

33. The method of claim 30 including the step of selecting the consumable oil, either singly or in combination from the group comprising animal oils, vegetable oils, marine-based oils, and algae oils.

34. The method of claim 30 including the step of selecting the at least one nutrient from the group comprising omega fatty acids, vitamin D, Co Q10, green tea extract, and policosanol.
